(19) United States
(12) Patent Application Publication
(10) Pub. No.: US 2007/0071701 A1
(43) Pub. Date: Mar. 29, 2007

(54) Antiperspirant actives and compositions

(76) Inventors: John Alan Hough, Bebington (GB); Gordon George McLeod, Bebington (GB); Jonathan Osler, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/547,110

(22) PCT Filed: Feb. 17, 2004
(86) PCT No.: PCT/EP04/01562

§ 371 Date: Nov. 24, 2006

(30) Foreign Application Priority Data
Feb. 27, 2003 (GB) 0304508.5

(51) Int. Cl. A61K 8/31

(52) U.S. Cl. 424/65

(57) ABSTRACT

Polyol-sprayed particulate antiperspirant actives, methods of manufacture thereof, and compositions comprising polyolsprayed particulate antiperspirant activities and carrier materials are described.

The present invention relates to antiperspirant actives, their method of preparation, and compositions comprising antiperspirant actives.

Typical antiperspirant compositions on the market use aluminium or aluminum-zirconium salts to prevent, or at least control, perspiration on the skin, particularly in the underarm, whilst generally simultaneously providing a deodorancy benefit.

A disadvantage of many antiperspirant compositions is their perceived skin unfriendliness. More particularly, the use of typical antiperspirant actives that are astringent metal salts is perceived to have a drying and tightening effect on the user's skin following application, resulting in dry skin, reduced skin elasticity and an unpleasant skin sensation.

Many antiperspirant actives can also result in a stinging sensation on the skin following application. Stinging is particularly problematic when an antiperspirant is applied following shaving.

In order to overcome these disadvantages, moisturising creams have been incorporated into antiperspirant compositions as, for example, described in U.S. Pat. No. 5,932,199, U.S. Pat. No. 6,099,827, and U.S. Pat. No. 6,221,345.

Polyols, in particular glycerol, are preferred components of moisturising creams. However, the incorporation of such materials into antiperspirant compositions comprising particulate antiperspirant active can cause problems, particularly at high levels of incorporation. One of the more significant problems is grit formation during the manufacture of the composition, which can noticeably impair the sensory properties of the composition and, particularly with spray dispensers, leading to blockage of the dispenser. This problem was addressed in U.S. Pat. No. 5,932,199 by adding further components during formulation; however, it would clearly be better if the problem could be solved without the use such additional processing aids.

Polyol-containing particulate antiperspirant actives have been produced by spray-drying solutions comprising both polyol and antiperspirant active as, for example, described in U.S. Pat. No. 6,451,296. However, the heating involved in such processes can lead to the production of one or more undesirable by-products. Compositions including such antiperspirant actives are disclosed in WO 03/070210.

It is an object of the present invention to provide an antiperspirant composition comprising a polyol that has excellent aesthetic properties.

It is a further object of the present invention to provide an antiperspirant composition comprising a polyol that does not require the use of additional processing aids.

It is a further object of the present invention to provide an antiperspirant active comprising a polyol that does not require heating of the antiperspirant active with the polyol.

It is a further object of the present invention to provide an antiperspirant composition containing a polyol that does not feel gritty.

It is a further object of the present invention to provide an antiperspirant composition containing a polyol that has improved thermal stability.

According to a first aspect of the invention, there is provided an antiperspirant composition comprising a polyol-sprayed sprayed particulate antiperspirant active and a carrier material.

According to a second aspect of the invention, there is provided a polyol-sprayed sprayed particulate antiperspirant active.

According to a third aspect of the invention, there is provided a method of manufacture of an antiperspirant active comprising spraying a particulate antiperspirant active with a polyol.

According to a fourth aspect of the invention, there is provided a method of manufacture of an antiperspirant composition comprising the suspension of an antiperspirant active as described in the second aspect of the invention in a carrier material.

In the present invention it has been found that suspending polyol-sprayed sprayed particulate antiperspirant actives in antiperspirant compositions leads to numerous benefits.

The benefits gained include having improved sensory properties, in particular, not feeling gritty, being easy to apply, having good glide, and feeling lighter. Compositions according to the invention can have improved physical properties, including any of the following: being easier to process, easier to apply, having improved thermal stability, and having better absorption. Spray compositions according to the invention have the additional benefit of reduced blockage of the narrow orifices of applicators, in particular nozzles, used with such compositions.

The antiperspirant compositions of the invention are typically anhydrous, meaning that they contain less than 10% water, and preferably less than 5% water. The water content includes any water complexed with and forming part of the antiperspirant active. The antiperspirant composition comprises a carrier material and may be suitable for application by direct contact with the human body and/or may be suitable for spray application.

The polyol-sprayed particulate antiperspirant active is generated by the polyol being sprayed onto the surfaces of antiperspirant active particulates. It is preferred that a polyol that is liquid at 25° C. is used. The polyol-sprayed particulate antiperspirant active is, in general, a particulate antiperspirant active permeated by the polyol; that is to say, the polyol is absorbed by the antiperspirant active and exists within it. Typically, the polyol is not evenly distributed throughout the individual antiperspirant particulates; usually, the polyol is present at a higher concentration at the surface of the particulates than at their centres. The surface of the particulates should be understood to mean that layer of the particulates representing the outer 25%, in particular the outer 10%, of the volume of the particulates.

The weight ratio of polyol to antiperspirant active in the polyol-sprayed particulate antiperspirant active may be 1:99 or greater, in particular 5:95 or greater, and especially 1:9 or greater, although the ratio is generally not greater than 1:4.

The particle size distribution of the polyol-sprayed antiperspirant active is typically such that 95%, preferably 99%, can pass through a 125 micron sieve. It is further preferred that the median particle size is less than 25 microns. In compositions comprising an aluminium-zirconium antiperspirant active (vide infra), it is preferred that the median particle size is less than 5 microns. Throughout this specification, references to median particle or droplet sizes should be understood to refer to D50 median volume values, as can be determined using standard light scattering methods with equipment such as the Malvern Mastersizer.

Suitable polyols for the present invention are generally not polymers, polymeric polyols typically being difficult to apply, poorly absorbed by the antiperspirant active, and generally giving reduced benefits. Preferred polyols, giving superior benefits, are polyhydric aliphatic alcohols, in particular those having from 2 to 8 carbon atoms, especially from 3 to 6 carbons atoms, and from 2 to 8 hydroxyl groups, especially from 3 to 6 hydroxyl groups. Specifically, suitable polyols which may be useful for the present invention include propylene glycol, butanetriol, glycerol, pentaerythritol, hexane-1,2-diol, sorbitol, xylitol, dulcitol, mannitol, mesoerythritol, trimethylolpropane, adonitol, arabitol, threitol, inositol, scyllitol, iditol, 2,5-anhydro-D-mannitol, 1,6-anhydro-glucose, and hexanetriol. Most preferably, the polyol is glycerol. The preferred polyols generally give superior sensory benefits.

Suitable antiperspirant actives are astringent metal salts. The astringent salts may be inorganic or organic salts of aluminum, zirconium, zinc and mixtures thereof. Actives useful as astringents or as components of astringent aluminum complexes include aluminum halides, aluminum hydroxyhalides, zirconyl oxyhalides, zirconyl hydroxyhalides and mixtures of these active materials.

Aluminum salts of this type include aluminum chloride and the aluminum hydroxyhalides having the general formula Al₂(OH)ₓQ_{y}-XH₂O where Q is chlorine, bromine or iodine, where x is 2 to 5 and x+y=6 and x and y do not need to be integers; and where X is about 1 to 6.

Several types of complexes utilizing the above astringent salts are known in the art. For example, U.S. Pat. No. 3,792,068 discloses complexes of aluminum, zirconium and amino acids such as glycine. Complexes reported therein and similar structures are commonly known as ZAG. The ZAG complexes ordinarily have an Al:Zr ratio of from about 1.67 to 12.5 and a metal:Cl ratio of from about 0.73 to 1.93.

Antiperspirant actives which are particularly useful for the present invention may be selected from aluminum chloride, aluminum chlorohydrate (ACH), aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum sulfate, aluminum zirconium octachlorohydrate, aluminum zirconium octachlorohydrex GLY, aluminum zirconium pentachlorohydrate, aluminum zirconium pentachlorohydrex GLY, aluminum zirconium tetrachlorohydrate, aluminum zirconium trichlorohydrate, aluminum zirconium tetrachlorohydrate GLY, and aluminum zirconium trichlorohydrate GLY.

Especially preferred antiperspirant actives are ones that have undergone some form of ‘activation’ process. Thus, activated ACH (‘AACH’) is an especially preferred active, as is activated ZAG (‘AZAG’).

For use in spray compositions, antiperspirant active comprising aluminium but not zirconium is preferred.

The amount of water associated with the antiperspirant active, excluding its associated polyol, that may be removed by standard methods and measured using a laboratory infrared moisture balance is preferably less than 10%, particularly less than 8%, and especially from 6 to 7% by weight of the active (including associated water).

The method of manufacture of the polyol-sprayed particulate antiperspirant active may involve spraying of a particulate antiperspirant active with a neat polyol or with a solution thereof. It is preferred that the amount of solvent used in minimised, for the reason of process simplicity. The use of a neat polyol is most preferred; however, solutions of polyol concentration of greater than 50%, 25%, or 10% (by weight) may alternatively be employed with decreasing preference. It is further preferred that the spraying be performed at ambient temperature. Clearly, when ambient temperature spraying of a neat polyol is performed, it is required that the polyol be a liquid at this temperature.

The particle size distribution of the antiperspirant active feedstock, i.e. prior to spraying, is typically such that 95%, preferably 99%, can pass through a 125 micron sieve. It is further preferred that the median particle size is less than 25 microns. In compositions comprising an aluminium-zirconium antiperspirant active (vide infra), it is preferred that median particle size is less than 5 microns. Prior to spraying, it is preferred that the antiperspirant active is dried in order to reduce the water content (vide supra); this can give benefits resulting from reduced agglomeration.

The antiperspirant active is generally subjected to agitation during the polyol spraying process; this can improve the efficiency of the spraying process. Good results are obtained when the polyol is sprayed onto a fluidised bed of particulate antiperspirant active. The latter process is typically performed on a perforated bed, through which air is projected into the active, in particular from underneath, thereby causing agitation of the active and ultimately suspending it in a stream of air. The inlet air used to fluidise the antiperspirant active is preferably pre-treated to remove water. This may be done by cooling to 5 to 10° C. below ambient temperature, removing condensed water, and then optionally warming or allowing to warm to ambient temperature. Throughout this specification, ambient temperature should be understood to be typically from 18 to 30° C.

The polyol is typically sprayed onto the antiperspirant active from a nozzle or plurality of nozzles. The polyol spray generally has a median droplet size of less than 200 microns, in particular less than 100 microns. It is preferred that the median droplet size of the spray is less than an order of magnitude different to the median particle of the particulate antiperspirant active feedstock. In some embodiments, it is preferred that the ratio of the median droplet size of the spray to the median particle size of the antiperspirant active feedstock is from 1:5 to 5:1.

The antiperspirant active may have a processing aid present when it is sprayed with the polyol. Preferred processing aids have an affinity for the polyol; examples include fumed silica, calcite, zeolite MAP, and finely ground zeolite 4A. Such processing aids may be present at from 0.05% to 100%, in particular from 1% to 25%, and especially at from 2% to 20% by weight, relative to the level of antiperspirant active (excluding the associated polyol, but including any associated water of hydration).

Immediately after spraying with the polyol and prior to any subsequent processing step, it is preferred that the median particle size does not increase to greater than 400 microns, it is particularly preferred that the median particle size does not increase to greater than 10 times its original value.

Subsequent to the application of the polyol, the antiperspirant active may be milled to reduce its median particle size. In certain embodiments, the median particle size is reduced to a value of from 80 to 120%, in particular from 90 to 110% of that of the antiperspirant active feedstock (vide supra). Milling can lead to superior benefits, including reduced blockage of orifices in spray applicators.

In certain embodiments, the median particle size of the antiperspirant active feedstock may be smaller than that of the antiperspirant active after spraying and optional subsequent milling; in particular, it may be less than half, and especially less than one third, that of the active after spraying and optional subsequent milling. The median particle size of the antiperspirant active feedstock may be less than 15 microns, in particular less than 10 microns, and especially less than 5 microns.

The antiperspirant active, excluding its associated polyol, but including any associated water of hydration, may be present at from 1 to 40%, preferably at from 5 to about 35%, and optimally at from 10 to 30% by weight of the composition.

Antiperspirant compositions in accordance with the present invention may be made in various forms and may be suitable for direct application (e.g. solid sticks, soft solids, creams, and roll-ons) or spray application (e.g. aerosol compositions). It is essential that the composition comprises a polyol-sprayed particulate antiperspirant active and a carrier material. The compositions in accordance with the present invention are suspensions of the particulate antiperspirant active in the carrier material.

Spray compositions are preferred embodiments of the present invention. Such compositions can deliver superior sensory benefits (vide supra) and the polyol-sprayed particulate antiperspirant active gives the additional benefit of reduced blockage of the applicator. Spray compositions comprise a continuous phase that is liquid at ambient temperature, although they may comprise a volatile component (e.g. propellant) with a boiling point of below ambient temperature that is kept in a liquid state by use of elevated pressure. Spray compositions comprising such a volatile component (i.e. aerosol compositions) are particularly advantageous embodiments of the present invention, valve blockage being a prevalent problem with such compositions when polyol inclusion is attempted without the use of the present invention.

Polyol additional to that associated with the active may be present in the composition. The total amount of polyol present in the composition may be from 0.1 to 5%, preferably from 0.5 to 3%, and optimally from 1 to 2.5% by weight.

The carrier material may be any component or combination of components that is capable of delivering the polyol-sprayed antiperspirant active to the skin. It may be present at from 1 to 99%, in particular from 50 to 98%, and especially from 60 to 95% by weight of the composition.

A suitable component of the carrier material is a volatile carrier fluid. Examples of volatile carrier fluids include volatile liquid siloxanes, in particular polyorganosiloxanes having a measurable vapour pressure at ambient conditions. The volatile polyorganosiloxanes may be linear or cyclic or mixtures thereof. Preferred volatile polyorganosiloxanes include polydimethylsiloxanes, in particular those containing from 3 to 9 silicon atoms and preferably not more than 7 silicon atoms. Particularly preferred polydimethylsiloxanes are cyclic and contain from 4 to 6 silicon atoms, otherwise often referred to as cyclotetramethicone, cyclopentamethicone and cyclohexamethicone, and mixtures thereof.

Examples of commercially available volatile liquid siloxanes include DC 344, DC 345 and DC 244, DC 245, DC 246, and grades of DC 200 with viscosity of below 10 centistokes (all from Dow Corning Corporation); Silicone 7207 and Silicone 7158 (from Union Carbide Corporation); and SF1202 (from General Electric [US]). Volatile liquid silicones are often present in antiperspirant compositions in a proportion of from 10 to 90% and in many compositions from 20 to 70% by weight of the composition, excluding any volatile propellant that might be present.

A suitable component of the carrier material is an emollient oil, often present at from 0.5 to 80%, in particular from 1 to 30% by weight of the composition. When a volatile liquid siloxane is also present, the weight ratio of emollient oil (excluding any volatile liquid siloxane emollient) to volatile liquid siloxane is preferably from 1:3 to 1:100. Suitable emollient oils include silicone oils and oils having a hydrocarbon backbone (including hydrocarbon backbones interrupted by one or more ester or ether groups). Emollient silicone oils are preferred in some embodiments, particularly at a level of from 1 to 15% by weight of the composition. Examples include polyalkyl siloxanes, polyalkylaryl siloxanes and polyethersiloxane copolymers, particularly dimethicone and dimethicone copolyols. Commercially available silicone emollient oils include grades of DC 556 and DC 200 series having viscosities of above 20 centistokes (from Dow Corning).

Emollient oils having a hydrocarbon backbone are preferred in certain embodiments. Ester oils are preferred in some of these embodiments, examples including liquid aliphatic esters, such as esters derivable from C₁-C₂₀ alkanols esterified with a C₈ to C₂₂ alkanoic acid or C₆ to C₁₀ alkanedioic acid. Suitable aliphatic esters include isopropyl myristate, lauryl myristate, isopropyl palmitate, diisopropyl sebacate and diisopropyl adipate, of which isopropyl palmitate is particularly favoured. Further suitable ester oils comprise liquid aromatic esters, including fatty alkyl benzoates having a melting point of below 20° C., such as C₈ to C₁₈ alkyl benzoates.

In some embodiments, a preferred component is a liquid aliphatic hydrocarbon such as a mineral oil, hydrogenated polyisobutene, polydecene, or liquid paraffin/isoparaffins containing at least 10 carbons.

In some embodiments, a preferred component is a liquid aliphatic ether derivable from at least one C₈ to C₁₈ fatty alcohol, particularly polyglycol ethers, such as PPG-3 myristyl ether or lower alkyl (C₄ to C₁₀) ethers of polyglycols such as PPG-14 butyl ether.

In spray compositions according to the invention, the carrier material will generally comprise a volatile propellant. Suitable volatile propellants include volatile organic compounds of boiling point less than 40° C., preferably less than 20° C., and optimally no higher than 10° C. Particular classes include C₁-C₆ hydrocarbons, C₂-C₈ dialkyl ethers, carbon dioxide and halo hydrocarbons. Among the useful C₁-C₆ hydrocarbons are propane, isopropane, butane, isobutane, isopentane, pentane and mixtures thereof. Propellants are available under the mark A31 (purely isobutane) and A45 (isobutane/isopropane) from the Phillips Petroleum Company. A preferred propellant is A50 which is a blend of isobutane/propane. Another useful propellant is dimethyl ether. In compositions comprising volatile propellant, this component is typically present at from 40 to 99%, and particularly at from 50 to 95% by weight of the composition.

In compositions according to the invention taking the form of solid sticks, soft solids, creams, and sometimes roll-ons, the carrier material will often comprise a thickener and/or structurant. Such materials may enhance many of the properties of the compositions of the invention, in particular those relating to sensory benefits. Organic structurants and/or inorganic thickeners may be used. Examples include hydrogenated vegetable oil, hydrogenated castor oil, fatty acids, beeswax, paraffin wax, silicone wax, fatty alcohols, polymers such as hydroxypropylcellulose, clays such as bentone, natural or synthetic gums, or mixtures or combinations thereof.

Organic structurants may be non-polymeric, for example fatty acids or salts thereof, typically having from 12 to 30 carbon atoms (e.g. stearic acid or sodium stearate) or fatty alcohols, typically being insoluble in water and generally having from 12 to 30 carbon atoms (e.g. stearyl alcohol, behenyl alcohol, or 12-hydroxystearic acid fatty). Other non-polymeric organic structurants that may be employed include esters and amides of C₁₂ to C₃₀ fatty acids; sterols, such as lanosterol; dibenzoyl alditols, such as dibenzoyl sorbitol; and certain organic waxes.

An organic wax is preferred component in some compositions according to the invention. Examples include paraffin wax, microcrystalline wax, ceresin, squalene, and polyethylene wax (molecular weight typically 200 to 10000); wax derived or obtained from plants or animals such as hydrogenated castor oil (castor wax), carnabau, spermacetti, candelilla, beeswax, modified beeswax, Montan wax and individual waxy components thereof, C₁₂ to C₃₀ fatty ester derivatives of polyols, in particular C₁₂ to C₃₀ fatty ester derivatives of glycerol, especially di- and tri-glycerides (synthetic versions being available as various grades of Synchrowax™) Organic wax structurants may be present in an amount of from 5 to 20% by weight of the composition.

Mixtures of organic structurants may be employed, such as mixtures of a fatty acid/salt with an organic wax.

Some suitable organic structurants form a fibrous network, such as selected n-acyl amino acid derivatives, including ester and amide derivatives, such as N-Lauroyl-L-glutamic acid di-n-butylamide, either by itself or when contemplated in conjunction with hydroxystearic acid or an ester or amide derivative thereof. Still further gellants include amide derivatives of di or tribasic carboxylic acids, such as alkyl N,N′ dialkylsuccinimides, eg dodecyl N,N′-dibutylsuccinimide.

Organic structurants may be polymeric, for example organo-polysiloxane elastomers, otherwise known as silicone gels, such as reaction products of a vinyl terminated polysiloxane and a cross linking agent or alkyl or alkyl polyoxyalkylene-terminated poly(methyl substituted) or poly(phenyl substituted) siloxanes. Other suitable polymeric structurants may be selected from polyamides, such as Versamid™; polyacrylamides; polysiloxane/polyamide copolymers; styrene/alkylene block copolymers, such as Kraton G™; styrene copolymers, such as Kristalex™; and C₁₂ to C₁₈ fatty acid esters of polysaccharides, such as dextrin (especially with 10 to 50 repeat units), or cellulosic polymers. Polymeric structurants are often employed in an amount of from 1 to 15%, in particular from 2 to 10%, and especially from 3 to 7% weight of the composition.

Where a monohydric alcohol and/or a polyol comprises a significant fraction of the carrier material, a dibenzoyl derivative of a saccharide, especially dibenzoyl sorbitol, is a preferred additional component.

Where a volatile liquid siloxane comprises a significant fraction of the carrier material, a silicone elastomer, especially a crosslinked polyorganosiloxane, is a preferred additional component, especially at a weight ratio to the volatile liquid siloxane of from 1:3 to 1:20.

Inorganic thickeners are often selected from siliceous and alumino-siliceous materials including silicas and clays. Many inorganic thickeners comprise a particulate colloidal silica, usually having a small particle size, such as below 1 μm. When used as the primary thickener, it is normally present in an amount of at least 3% by weight and particularly 4 to 7% by weight. It can be used as a supplementary thickener in lower amounts such as up to 3% by weight.

Optionally, additional components may be used. Perfume is a frequently used additional component. Normally it is incorporated within an oily phase in the composition, and typically is present in an amount of from 0.1 to 5%, in particular from 0.2 to 2.5% by weight of the composition. The perfume can be introduced in its natural form, i.e. normally as an oil, or it can be wholly or partially encapsulated.

Additional bulking agents/fillers may be employed. Examples are typically particulate fillers, for example talc, sodium bicarbonate, starch, including cornstarch, modified starch, and mixtures thereof. The amount of such additional fillers/bulking agents is often not more than 15%, and is preferably up to 10%, especially 1 to 5% by weight of the composition.

Suspending agents, for example clays and/or silicas, may be employed in compositions according to the invention. Such ingredients may also function as processing aids (vide supra). Suitable clays include bentonites, hectorites and colloidal magnesium aluminium silicates. Commercially available clays are available under the trademarks Veegum and Laponite. It is preferable to include montmorillonite clays which have been hydrophobically surface treated, for example by reaction with an amine. Preferred hydrophobic-treated clays are available under the Trademark Bentone (various grades). Fumed silica is a preferred suspending agent. Suspending agents may be present at from 0.05% to 100%, in particular from 1% to 25%, and especially at from 2% to 20% by weight, relative to the level of antiperspirant active (excluding the associated polyol, but including any associated water of hydration). Based on the total weight of composition excluding any volatile propellant that may be present, suspending agents may be present at up to 15%, particularly up to 10%, and especially at from 1 to 5% by weight.

Additional wash-off agents may be employed, such components serving to assist in the removal of the composition from skin or clothing. Such wash-off agents are typically nonionic surfactants such as esters or ethers containing a C₈ to C₂₂ alkyl moiety and a hydrophilic moiety which can comprise a polyoxyalkylene group (POE or POP) and/or a polyol. Such components may be used in an amount of up to 10% by weight of the composition.

Other optional components include anti-clogging agents (for compositions in the form of fluids suitable for spray application); additional skin benefit agents; preservatives; and colouring agents.

### EXAMPLES

Throughout these Examples, polyol levels are expressed as weight percentage on antiperspirant active, i.e. weight percentage of polyol relative to the weight of active excluding any associated polyol.

### Examples 1 to 3

### Preparation of Glycerol-Sprayed AACH

The glycerol-sprayed particulate AACH actives described in Table 1 were prepared in the following manner.

10 kg of AACH antiperspirant powder (A296, ex BK Giulini Chemie GmbH) was loaded into a cylindrical fluid bed of 0.202 m diameter. The flow of inlet air through the base plate was gradually increased until the powder reached a fluidised state. The inlet air was pre-treated to remove some moisture by cooling to 15° C. and then warming to ambient temperature (ca. 20° C.). Glycerol, at the level indicated in Table 1, was sprayed onto the fluidised powder via a twin phase (liquid/air) SUE25 nozzle (ex Spray Systems Inc.), with air atomisation using an air pressure of 4 bar.

The actives resulting from the first step of the process as described above were transferred to a 100AFG fluidised bed jet mill (ex Hosokowa Micron Ltd.) and milled at 2250 rpm for the times indicated in Table 1. Each of the particulate actives resulting was of good quality: for each one greater than 99% by weight passed through a 125 micron sieve and the median particle size was less than 25 microns.

TABLE 1
Glycerol-sprayed AACH samples

| | Glycerol | | Wt. % | Median |
|---|---|---|---|---|
| | (wt % on | Milling time | retained by | particle |
| Example | AACH) | (mins.) | 125μ sieve | size (μ) |
| 1 | 10 | 90 | Trace | 21.2 |
| | | | | 20.4 |
| 2 | 15 | 119 | Trace | 22.2 |
| | | | | 22.3 |
| 3 | 20 | 121 | 0.4% | 23.4 |
| | | | | 22.7 |

Duplicate determinations of the median particle size were made on a Malvern Mastersizer instrument.

### Examples 4 to 6

### Preparation of Glycerol-Sprayed AZAG

The glycerol-sprayed particulate AZAG actives described in Table 2 were prepared in a manner analogous to the AACH activities described in Table 1. Again, good quality actives were produced; single particle size determinations being made for these samples.

TABLE 2
Glycerol-sprayed AZAG samples

| | Glycerol (wt % on | Milling time | Median particle |
|---|---|---|---|
| Example | AZAG) | (min.) | size (μ) |
| 4 | 10 | 113 | 3.6 |
| 5 | 15 | 122 | 3.4 |
| 6 | 20 | 180 | 3.5 |

### Examples 7 to 9

Compositions as described in Table 3 may be prepared using polyol-sprayed antiperspirant actives as described hereinbefore. All percentages are by weight and the levels of antiperspirant active indicated include the polyol associated therewith (e.g. glycerol at 10% on weight of the antiperspirant active). Examples 7 and 8 are aerosol compositions and Example 9 is a solid stick composition.

TABLE 3
Example Compositions

| | Example: | | |
|---|---|---|---|
| | 7 | 8 | 9 |
| Components: | % w/w | | |
| Bentone 38V | 0.50 | 1.00 | — |
| Fragrance | 1.00 | 0.50 | 1.20 |
| PPG-14 butyl ether | 3.00 | 6.00 | 16.50 |
| PEG-8 distearate | — | — | 2.00 |
| Castor wax | — | — | 2.00 |
| Octadecanol | | | 14.50 |
| Octyl dodecanol | 0.10 | 0.20 | — |
| Preservative | — | 0.10 | 0.05 |
| Talc | — | — | 2.00 |
| Fumed silica | — | 0.05 | — |
| AZAG | — | — | 28.00 |
| AACH | 4.90 | 10.00 | — |
| Dimethiconol | 0.20 | 0.20 | — |
| Cyclomethicone | 3.20 | 6.80 | To 100 |
| CAP 40 (propellant) | To 100 | To 100 | — |

1. An antiperspirant composition comprising a polyol-sprayed particulate antiperspirant active and a carrier material.

2. An antiperspirant composition according to claim 1, wherein the weight ratio of polyol to antiperspirant active is 1:99 or greater.

3. An antiperspirant composition according to claim 1, wherein the weight ratio of polyol to antiperspirant active is 1:9 or greater.

4. An antiperspirant composition according to claim 1, suitable for application by direct contact with the human body.

5. An antiperspirant spray composition according to claim 1.

6. An antiperspirant spray composition according to claim 5, that is an aerosol composition.

7. A polyol-sprayed particulate antiperspirant active, wherein the polyol is not a polymer.

8. An antiperspirant active according to claim 7, wherein greater than 95% by weight can pass through a 125 micron sieve.

9. An antiperspirant active according to claim 8, having a median particle size of less than 25 microns.

10. An antiperspirant active according to claim 7, wherein the polyol is glycerol.

11. A method of manufacture of an antiperspirant active comprising spraying of a particulate antiperspirant active with a polyol.

12. A method according to claim 11, comprising spraying a particulate antiperspirant active with a neat polyol or a solution of polyol concentration of greater than 25% by weight.

13. A method according to claim 12, comprising spraying a particulate antiperspirant active with a neat polyol.

14. A method according to claim 11, comprising spraying of a particulate antiperspirant active at ambient temperature.

15. A method according to claim 11, comprising spraying a polyol onto a fluidised bed of particulate antiperspirant active.

16. A method according to claim 15, wherein air that has been pre-treated to remove moisture is used to fluidise the particulate antiperspirant active.

17. A method according to claim 11, comprising a subsequent milling step to reduce the median particle size of the antiperspirant active.

18. A method of manufacture of an antiperspirant composition comprising the suspension of an antiperspirant active as described in claim 7 in a carrier material.
